(19) United States
(12) Patent Application Publication
(10) Pub. No.: US 2026/0038091 A1
(43) Pub. Date: Feb. 5, 2026

(54) IMAGE PROCESSING METHOD, IMAGE PROCESSING DEVICE, PROGRAM

(71) Applicant: NIKON CORPORATION, Tokyo (JP)

(72) Inventors: Mariko HIROKAWA, Yokohama-shi (JP); Yuanting JI, Yokohama-shi (JP)

(21) Appl. No.: 19/355,224

(22) Filed: Oct. 10, 2025

Related U.S. Application Data
(63) Continuation of application No. PCT/JP2024/014452, filed on Apr. 9, 2024.

(30) Foreign Application Priority Data
Apr. 11, 2023 (JP) 2023-064527

(51) Int. Cl. G06T 5/50; A61B 3/10; A61B 3/12; G06T 5/94

(52) U.S. Cl. CPC G06T 5/50; A61B 3/102; A61B 3/1241; G06T 5/94; G06T 2207/10101; G06T 2207/10121; G06T 2207/20212; G06T 2207/30041; G06T 2207/30101

(57) ABSTRACT

An image processing method performed by a processor includes: a step of acquiring a fundus image in which choroid blood vessels are visualized; a step of extracting choroid arteries in the fundus image acquired in the acquiring step by performing image processing on the fundus image; and a step of generating a fundus image in which the choroid arteries extracted in the extracting step are highlighted.

### CROSS-REFERENCE TO RELATED APPLICATION

This application is a continuation application of International Application No. PCT/JP2024/014452, filed Apr. 9, 2024, the disclosure of which is incorporated herein by reference in its entirety. Further, this application claims priority from Japanese Patent Application No. 2023-064527, filed Apr. 11, 2023, the disclosure of which is incorporated herein by reference in its entirety.

### TECHNICAL FIELD

The present disclosure relates to an image processing method, an image processing device, and a program.

### BACKGROUND ART

U.S. Pat. No. 8,356,901 discloses technology to analyze vortex veins from fundus images. Moreover, Japanese Patent Application Laid-Open (JP-A) No. 2015-131 discloses technology to quantify choroid blood vessels from measurement data of optical coherence tomography (OCT).

### SUMMARY OF INVENTION

### Technical Problem

A first aspect of the present disclosure is an image processing method performed by a processor. The image processing method includes a step of acquiring a fundus image in which choroid blood vessels are visualized, a step of extracting choroid arteries in the fundus image acquired in the acquiring step by performing image processing on the fundus image; and a step of generating a fundus image in which the choroid arteries extracted in the extracting step are highlighted.

A second aspect of the present disclosure is an image processing device including an acquisition unit configured to acquire a fundus image in which choroid blood vessels are visualized, an extraction unit configured to extract choroid arteries in the fundus image acquired by the acquisition unit by performing image processing on the fundus image; and a generation unit configured to generate a fundus image in which the choroid arteries extracted by the extraction unit are highlighted.

A third aspect of the present disclosure is a computer executable program including a step of acquiring a fundus image in which choroid blood vessels are visualized, a step of extracting choroid arteries in the fundus image acquired in the acquiring step by performing image processing on the fundus image, and a step of generating a fundus image in which the choroid arteries extracted in the extracting step are highlighted.

### BRIEF DESCRIPTION OF DRAWINGS

FIG. 1 is a schematic configuration diagram of an ophthalmic system according to an exemplary embodiment.

FIG. 2 is a schematic configuration diagram of an ophthalmic device according to the present exemplary embodiment.

FIG. 3 is a schematic configuration diagram of a server.

FIG. 4 is an explanatory diagram of functions implemented by an image processing program in a CPU of a server.

FIG. 5 is a flowchart illustrating an example of a flow of image processing by a server.

FIG. 6 is a flowchart illustrating an example of a flow of fluoroscopic fundus contrast imaging analysis processing.

FIG. 7 is diagram illustrating an example of a fundus image of choroid blood vessels including choroid arteries.

FIG. 8 is an example of fundus images illustrated as a time series of fundus images around the time of contrast imaging.

FIG. 9A is a diagram illustrating an example of an image resulting from polar coordinate conversion of a fundus image.

FIG. 9B is a schematic diagram illustrating an example of setting regions in a fundus image.

FIG. 10 is a diagram illustrating an example of an image resulting from backwards conversion from a polar coordinate system image into a Cartesian coordinate system image.

FIG. 11 is a diagram illustrating an example of a display screen.

FIG. 12 is a flowchart illustrating an example of a flow of OCT analysis processing.

FIG. 13 is a schematic diagram of OCT volume data.

FIG. 14 is a schematic diagram illustrating a procedure up to choroid blood vessel extraction.

FIG. 15 is a flowchart illustrating an example of a flow of choroid blood vessel center position extraction processing.

FIG. 16 is a schematic diagram illustrating a procedure up to derivation of center lines of choroid blood vessels.

FIG. 17 is a schematic diagram related to an image in which plural en-face images have been combined.

FIG. 18 is a flowchart illustrating an example of a flow of processing to detect a second feature value of choroid blood vessels.

FIG. 19 is a diagram illustrating a relationship of analysis regions to a choroid blood vessel image.

FIG. 20 is a schematic diagram related to setting plural analysis regions.

FIG. 21 is a schematic diagram of analysis regions for choroid blood vessels.

FIG. 22 is a schematic diagram illustrating conditions of choroid blood vessels on the choroid in plural analysis regions.

FIG. 23 is a schematic diagram illustrating an example of an analysis region on a choroid region.

FIG. 24 is a diagram illustrating an example of a display screen.

FIG. 25 is a diagram illustrating an example of a display screen.

FIG. 26 is a diagram illustrating an example of a display screen.

FIG. 27 is an explanatory diagram related to separate presentation of a choroid blood vessel image and center lines.

### DESCRIPTION OF EMBODIMENTS

Detailed explanation follows regarding exemplary embodiments to implement technology disclosed herein, with reference to the drawings. Note that the same reference numerals are appended throughout the drawings to configuration elements and processing with the same operation, and performing the same function, and duplicate explanation thereof will be omitted as appropriate. Moreover, sometimes description will be omitted for configuration not directly related to the present disclosure and for known configuration. The dimensions and proportions in the drawings may be exaggerated for ease of explanation, and sometimes differ from actual proportions. Furthermore, each of the drawings is merely illustrated schematically to such a degree as to enable sufficient understanding of technology disclosed herein. Technology disclosed herein is accordingly not limited to the examples illustrated.

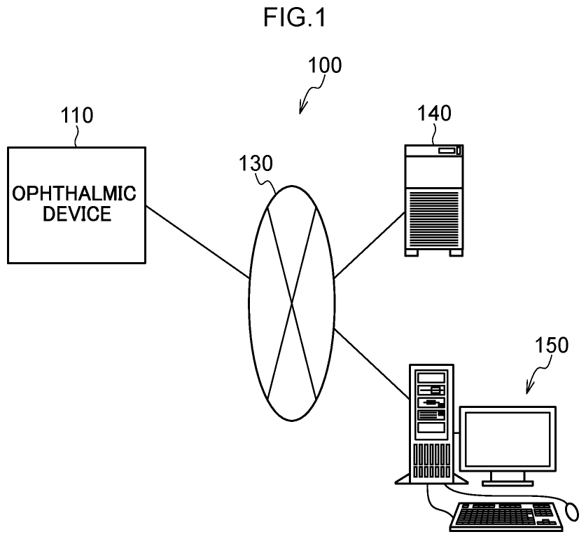

FIG. 1 illustrates a schematic configuration diagram of an ophthalmic system 100. As illustrated in FIG. 1, the ophthalmic system 100 includes an ophthalmic device 110, a server device (hereafter referred to as “server”) 140, and a display device (hereafter referred to as “viewer”) 150. The ophthalmic device 110 acquires fundus images. The server 140 stores plural fundus images obtained by imaging a fundus of plural respective patients using the ophthalmic device 110 and eye axial lengths measured using a non-illustrated eye axial length measurement device, with these being stored associated with respective patient IDs. The viewer 150 displays fundus images and analysis results acquired by the server 140.

The server 140 serves as an example of an “image processing device” of the present disclosure.

The ophthalmic device 110, the server 140, and the viewer 150 are connected together through a network 130. The network 130 is a freely selected network such as a LAN, WAN, the internet, a wide area Ethernet, or the like. For example, a LAN may be employed as the network 130 in cases in which the ophthalmic system 100 is built in a single hospital.

The viewer 150 is a client in a client-server system, and plural such devices are connected together through a network. There may also be plural devices for the server 140 connected through the network in order to provide system redundancy. Alternatively, if the ophthalmic device 110 is provided with image processing functionality and with the image viewing functionality of the viewer 150, then the fundus images may be acquired and image processing and image viewing performed with the ophthalmic device 110 in a standalone state. Moreover, if the server 140 is provided with the image viewing functionality of the viewer 150, then the fundus images may be acquired and image processing and image viewing performed by a configuration of the ophthalmic device 110 and the server 140.

Note that other ophthalmic equipment (examination equipment for measuring a field of view, measuring intraocular pressure, or the like) and/or a diagnostic support device that analyzes images using artificial intelligence (AI) may be connected to the ophthalmic device 110, the server 140, and the viewer 150 over the network 130.

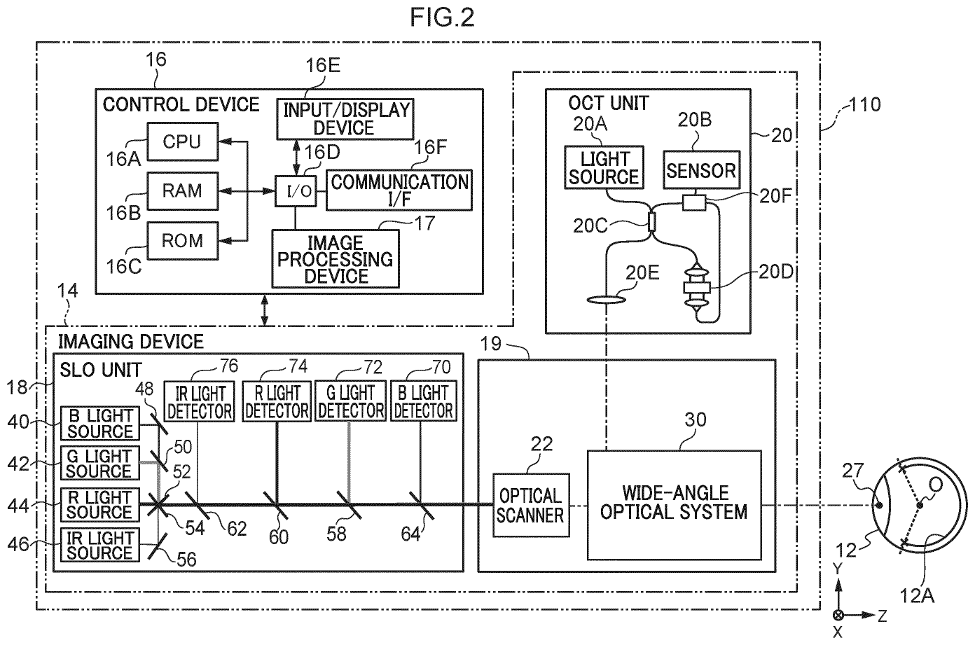

Next, explanation follows regarding a configuration of the ophthalmic device 110, with reference to FIG. 2.

For ease of explanation, scanning laser ophthalmoscope is abbreviated to SLO. Moreover, optical coherence tomography is abbreviated to OCT.

With the ophthalmic device 110 installed on a horizontal plane and a horizontal direction taken as an “X direction”, a direction perpendicular to the horizontal plane is denoted a “Y direction”, and a direction connecting the center of the pupil at the anterior eye portion of the examined eye 12 and the center of the eyeball is denoted a “Z direction”. The X direction, the Y direction, and the Z direction are thus mutually perpendicular directions.

The ophthalmic device 110 includes an imaging device 14 and a control device 16. The imaging device 14 is provided with an SLO unit 18, and an OCT unit 20, and acquires a fundus image of the examined eye 12. Two-dimensional fundus images that have been acquired by the SLO unit 18 are referred to as SLO images. Tomographic images, face-on images (en-face images) and the like of the retina generated based on OCT data acquired by the OCT unit 20 are referred to as OCT images.

The control device 16 includes a computer provided with a Central Processing Unit (CPU) 16A, Random Access Memory (RAM) 16B, Read-Only Memory (ROM) 16C, and an input/output (I/O) port 16D.

The control device 16 is provided with an input/display device 16E connected to the CPU 16A through the I/O port 16D. The input/display device 16E includes a graphical user interface to display images of the examined eye 12 and to receive various instructions from a user. An example of the graphical user interface is a touch panel display.

The control device 16 is also provided with an image processing device 17 connected to the I/O port 16D. The image processing device 17 generates images of the examined eye 12 based on data acquired by the imaging device 14. Note that the control device 16 is connected to the network 130 through a communication interface (I/F) 16F.

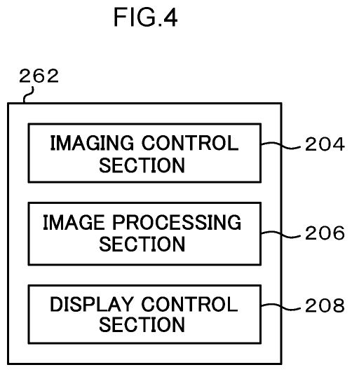

Although the control device 16 of the ophthalmic device 110 is provided with the input/display device 16E as illustrated in FIG. 2, the present disclosure is not limited thereto. For example, a configuration may adopted in which the control device 16 of the ophthalmic device 110 is not provided with the input/display device 16E, and instead a separate input/display device is provided that is physically independent of the ophthalmic device 110. In such cases, the display device is provided with an image processing processor unit that operates under the control of a display control section 208 (see FIG. 4) of the CPU 16A in the control device 16. Such an image processing processor unit may be configured so as to display SLO images and the like based on image signals output as instructed by the display control section 208.

The imaging device 14 operates under the control of the CPU 16A of the control device 16. The imaging device 14 includes the SLO unit 18, an imaging optical system 19, and the OCT unit 20. The imaging optical system 19 includes an optical scanner 22 and a wide-angle optical system 30.

The optical scanner 22 scans light emitted from the SLO unit 18 two dimensionally in the X direction and the Y direction. As long as the optical scanner 22 is an optical element capable of deflecting light beams, it may be configured by any out of, for example, a polygon mirror, a mirror galvanometer, or the like. A combination thereof may also be employed.

The wide-angle optical system 30 combines light from the SLO unit 18 with light from the OCT unit 20.

The wide-angle optical system 30 may be a reflection optical system employing a concave mirror such as an elliptical mirror, a refraction optical system employing a wide-angle lens, or may be a reflection-refraction optical system employing a combination of a concave mirror and a lens. Employing a wide-angle optical system that utilizes an elliptical mirror, wide-angle lens, or the like enables imaging to be performed not only of a central portion of the fundus, but also of the retina at the fundus periphery.

For a system including an elliptical mirror, a configuration may be adopted that utilizes an elliptical mirror system as disclosed in International Publication (WO) Nos. 2016/103484 or 2016/103489. The disclosures of WO Nos. 2016/103484 and 2016/103489 are incorporated in their entirety in the present specific by reference herein.

Observation of the fundus over a wide field of view (FOV) 12A is implemented by the wide-angle optical system 30. The FOV 12A refers to a range capable of being imaged by the imaging device 14. The FOV 12A may be expressed as a viewing angle. In the present exemplary embodiment, the viewing angle may be defined in terms of an internal illumination angle and an external illumination angle. The external illumination angle is the angle of illumination by a light beam shone from the ophthalmic device 110 toward the examined eye 12, and is an angle of illumination defined with respect to a pupil 27. The internal illumination angle is the angle of illumination of a light beam shone onto the fundus, and is an angle of illumination defined with respect to an eyeball center O. Correspondence relationships exist between the external illumination angle and the internal illumination angle. For example, an external illumination angle of 120° is equivalent to an internal illumination angle of about 160°. The internal illumination angle in the present exemplary embodiment is 200°.

SLO fundus images obtained by imaging at an imaging angle of view having an internal illumination angle of 160° or greater are referred to as UWF-SLO fundus images. UWF is an abbreviation of ultra-wide field (ultra-wide angled). A region extending from a posterior pole portion of a fundus of the examined eye 12 past an equatorial portion thereof can be imaged by the wide-angle optical system 30 having a field of view (FOV) angle of the fundus that is an ultra-wide field, enabling imaging of structural objects present at fundus peripheral portions.

The ophthalmic device 110 is capable of imaging a region 12A with an internal illumination angle of 200° with respect to a reference position of the eyeball center O of the examined eye 12. Note that an internal illumination angle of 200° corresponds to an external illumination angle of 110° with respect to the pupil of the eyeball of the examined eye 12 as the reference. Namely, the wide-angle optical system 30 images a fundus region with an internal illumination angle of 200° by shining laser light through the pupil at an angle of view external illumination angle of 110°.

An SLO system is realized by the control device 16, the SLO unit 18, and the imaging optical system 19 as illustrated in FIG. 2. The SLO system is provided with the wide-angle optical system 30, enabling fundus imaging over the wide FOV 12A.

The SLO unit 18 is provided with a blue (B) light source 40, a green (G) light source 42, a red (R) light source 44, an infrared (for example near infrared) (IR) light source 46, and optical systems 48, 50, 52, 54, 56 to guide the light from the light sources 40, 42, 44, 46 onto a single optical path using reflection and/or transmission. The optical systems 48, 56 are mirrors, and the optical systems 50, 52, 54 are beam splitters. B light is reflected by the optical system 48, is transmitted through the optical system 50, and is reflected by the optical system 54. G light is reflected by the optical systems 50, 54, R light is transmitted through the optical systems 52, 54, and IR light is reflected by the optical systems 52, 56. The respective lights are thereby guided onto a single optical path.

The SLO unit 18 is configured so as to be capable of switching between light sources for emitting laser light of different wavelengths or a combination of the light sources, such as a mode in which R light and G light are emitted, a mode in which infrared light is emitted, etc. Although the example in FIG. 2 includes four light sources, i.e. the B light source 40, the G light source 42, the R light source 44, and the IR light source 46, the present disclosure is not limited thereto. For example, the SLO unit 18 may also include a white light source, in a configuration in which light is emitted in various modes, such as a mode in which G light, R light, and B light is emitted, and a mode in which white light is emitted alone.

Light introduced to the imaging optical system 19 from the SLO unit 18 is scanned in the X direction and the Y direction by the optical scanner 22. The scanning light passes through the wide-angle optical system 30 and the pupil 27 and is shone onto the fundus. Reflected light that has been reflected by the fundus passes through the wide-angle optical system 30 and the optical scanner 22 and is introduced into the SLO unit 18.

The SLO unit 18 is provided with a beam splitter 64 and a beam splitter 58. From out of the light coming from a posterior eye portion (fundus) of the examined eye 12, the B light therein is reflected by the beam splitter 64 and light other than B light therein is transmitted by the beam splitter 64. From out of the light transmitted by the beam splitter 64, the G light therein is reflected by the beam splitter 58 and light other than G light therein is transmitted by the beam splitter 58. The SLO unit 18 is further provided with a beam splitter 60 that, from out of the light transmitted through the beam splitter 58, reflects R light therein and transmits light other than R light therein. The SLO unit 18 is further provided with a beam splitter 62 that reflects IR light from out of the light transmitted through the beam splitter 60. The SLO unit 18 is further provided with a B light detector 70 to detect B light reflected by the beam splitter 64, a G light detector 72 to detect G light reflected by the beam splitter 58, an R light detector 74 to detect R light reflected by the beam splitter 60, and an IR light detector 76 to detect IR light reflected by the beam splitter 62.

Light that has passed through the wide-angle optical system 30 and the optical scanner 22 and been introduced into the SLO unit 18 (i.e. reflected light that has been reflected by the fundus) is reflected by the beam splitter 64 and photo-detected by the B light detector 70 when B light, and is reflected by the beam splitter 58 and photo-detected by the G light detector 72 when G light. When R light, the incident light is transmitted through the beam splitter 58, reflected by the beam splitter 60, and photo-detected by the R light detector 74. When IR light, the incident light is transmitted through the beam splitters 58, 60, reflected by the beam splitter 62, and photo-detected by the IR light detector 76. The image processing device 17 that operates under the control of the CPU 16A employs signals detected by the B light detector 70, the G light detector 72, the R light detector 74, and the IR light detector 76 to generate UWF-SLO images.

UWF-SLO images generated using signals detected by the B light detector 70 are called B-UWF-SLO images (blue fundus images). UWF-SLO images generated using signals detected by the G light detector 72 are called G-UWF-SLO images (green fundus images). UWF-SLO images generated using signals detected by the R light detector 74 are called R-UWF-SLO images (red fundus images). UWF-SLO images generated using signals detected by the IR light detector 76 are called IR-UWF-SLO images (IR fundus images). UWF-SLO images encompass the red fundus images, the green fundus images, the blue fundus images, and the IR fundus images. Fluoroscopic UWF-SLO images imaged with florescent light are also encompassed therein.

The control device 16 also controls the light sources 40, 42, 44 so as to emit light at the same time. A green fundus image, a red fundus image, and a blue fundus image are obtained with mutually corresponding positions by imaging the fundus of the examined eye 12 at the same time with the B light, G light, and R light. An RGB color fundus image is obtained from the green fundus image, the red fundus image, and the blue fundus image. The control device 16 obtains a green fundus image and a red fundus image with mutually corresponding positions by controlling the light sources 42, 44 so as to emit light at the same time and imaging the fundus of the examined eye 12 at the same time with the G light and R light. An RG color fundus image is obtained from the green fundus image and the red fundus image. Moreover, a full color fundus image may be generated using the green fundus image, the red fundus image, and the blue fundus image.

A region extending from a posterior pole portion of a fundus of the examined eye 12 past an equatorial portion thereof can be imaged by the wide-angle optical system 30 with a field of view (FOV) angle of the fundus that is an ultra-wide field.

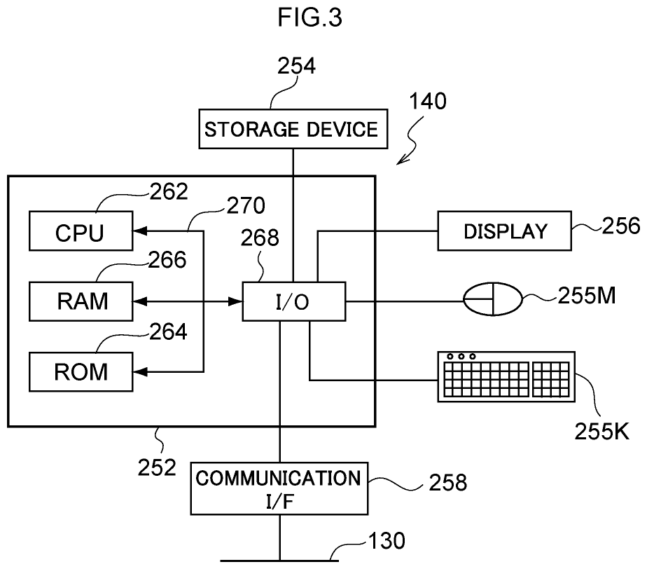

Image data of SLO images is sent from the ophthalmic device 110 to the server 140 though the communication interface 16F and is stored in a storage device 254 (FIG. 3).

An OCT system is implemented by the control device 16, the OCT unit 20, and the imaging optical system 19. The OCT system includes the wide-angle optical system 30, and is accordingly able to perform OCT imaging of fundus peripheral portions similarly to the imaging of SLO fundus image described above. Namely, OCT imaging over a region extending from a posterior pole portion of the examined eye 12 fundus past the equatorial portion is able to be performed by employing the wide-angle optical system 30 having a field of view (FOV) angle of the fundus that is an ultra-wide field. OCT data of structural objects such as choroid arteries present in the fundus peripheral portions can be acquired, and tomographic images of the choroid blood vessels, such as choroid arteries, and a 3D structure of the choroid blood vessels, such as choroid arteries, can be obtained by performing image processing on the OCT data.

The OCT unit 20 includes a light source 20A, a sensor (detection element) 20B, a first light coupler 20C, a reference optical system 20D, a collimator lens 20E, and a second light coupler 20F.

Light emitted from the light source 20A is split by the first light coupler 20C. One part of the split light is collimated by the collimator lens 20E into parallel light serving as measurement light before being introduced into the imaging optical system 19. The measurement light is shone onto the fundus through the wide-angle optical system 30 and the pupil 27. Measurement light that has been reflected by the fundus passes through the wide-angle optical system 30 so as to be introduced into the OCT unit 20, then passes through the collimator lens 20E and the first light coupler 20C before being incident to the second light coupler 20F.

The other part of the light emitted from the light source 20A and split by the first light coupler 20C is introduced into the reference optical system 20D as reference light, and is made incident to the second light coupler 20F through the reference optical system 20D.

The respective lights that are incident to the second light coupler 20F, namely the measurement light reflected by the fundus and the reference light, interfere with each other in the second light coupler 20F so as to generate interference light. The interference light is photo-detected by the sensor 20B. The image processing device 17 operating under the control of an image processing section 206 (see FIG. 4) generates OCT data detected by the sensor 20B. OCT images, such as tomographic images and en-face images, are able to be generated in the image processing device 17 based on this OCT data.

By employing the wide-angle optical system 30, the ophthalmic device 110 is able to employ the region 12A having an internal illumination angle of 200° as the scan target. Namely, OCT imaging is performed of the predetermined specific range by controlling the optical scanner 22. The ophthalmic device 110 is able to generate OCT data by this OCT imaging.

Thus the ophthalmic device 110 is able to generate OCT images such as tomographic images of the fundus (B-SCAN images), OCT volume data, and en-face images that are cross-sections of such OCT volume data (face-on images generated based on the OCT volume data). Note that OCT images obviously also encompass OCT images of a fundus center portion (posterior pole portion of the eyeball where the macular, the optic nerve head, and the like are present).

The OCT data (or image data of the OCT images) is sent from the ophthalmic device 110 to the server 140 though the communication interface 16F and is stored in a storage device 254 (see FIG. 3).

Note that although in the present exemplary embodiment an example is given in which the light source 20A is a wavelength swept-source OCT (SS-OCT), various types of OCT system may be employed, such as a spectral-domain OCT (SD-OCT) or a time-domain OCT (TD-OCT) system.

Next, description follows regarding a configuration of an electrical system of the server 140, with reference to FIG. 3. As illustrated in FIG. 3, the server 140 includes a computer main body 252. The computer main body 252 includes a CPU 262, RAM 266, ROM 264, and an input/output (I/O) port 268. The storage device 254, a display 256, a mouse 255M, a keyboard 255K, and a communication interface (I/F) 258 are connected to the input/output (I/O) port 268. The storage device 254 is, for example, configured by non-volatile memory. The input/output (I/O) port 268 is connected to the network 130 through the communication interface (I/F) 258. The server 140 is accordingly able to communicate with the ophthalmic device 110 and the viewer 150.

An image processing program is stored on the ROM 264 or the storage device 254.

The ROM 264 and the storage device 254 are examples of “memory” of the present disclosure. The CPU 262 is an example of a “processor” of the present disclosure. The image processing program is an example of a “program” of the present disclosure.

The server 140 stores respective data received from the ophthalmic device 110 in the storage device 254.

Description follows regarding various functions implemented by the CPU 262 of the server 140 executing the image processing program, with reference to FIG. 4. The image processing program executed by the CPU 262 includes an imaging control function, an image processing function, and a display control function, as illustrated in FIG. 4. By the CPU 262 executing the image processing program including each of these functions, the CPU 262 functions as an imaging control section 204, the image processing section 206, and a display control section 208.

The image processing section 206 is an example of an “acquisition unit”, “detection unit”, “extraction unit”, “generation unit”, and “estimation unit” of the present disclosure.

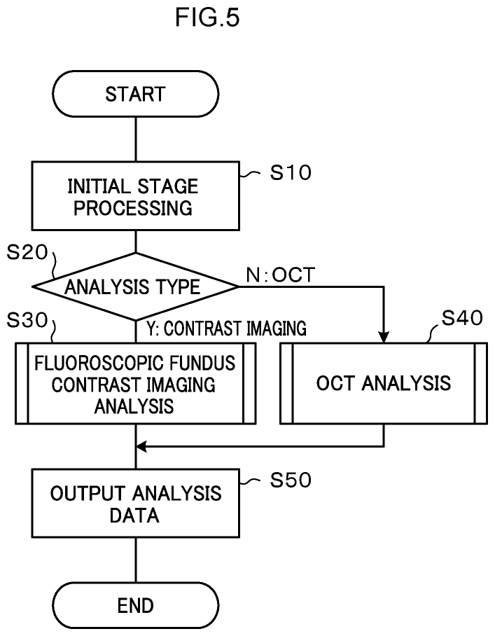

Next, detailed description follows regarding image processing executed by the server 140, with reference to FIG. 5. The image processing (image processing method) illustrated in FIG. 5 is implemented by the CPU 262 of the server 140 executing the image processing program.

The image processing program is executed when the server 140 has received an analysis start instruction, for example, on receipt of an analysis start instruction from operation of the keyboard 255K or the like of the server 140 by an operator, or an analysis start instruction from the viewer 150. Note that the image processing program may be executed when a fundus image imaged by the server 140 has been received from the ophthalmic device 110.

At step S10, an imaging control section 204 executes initial stage processing for analysis processing, described later. Initial setting of various parameters and the like is performed in the initial stage processing. This initial stage processing also includes processing to acquire information indicating an analysis processing type. In the present exemplary embodiment, description follows regarding a case in which the information indicating analysis processing type is information indicating one or other out of fluoroscopic fundus contrast imaging analysis, or OCT analysis. Note that at step S10, the imaging control section 204 may include processing to instruct the ophthalmic device 110 to perform imaging of a fundus image by SLO image imaging using the SLO unit 18, or by OCT image imaging using the OCT unit 20.

At step S20, the image processing section 206 determines whether or not the information indicating the analysis processing type indicates fluoroscopic fundus contrast imaging analysis, with processing proceeding to step S30 when determination is affirmative and processing proceeding to step S40 when determination is negative.

At step S30, the image processing section 206 executes fluoroscopic fundus contrast imaging analysis processing, at step S50 the display control section 208 outputs analysis data of the analysis result, and ends the present processing routine. On the other hand, at step S40, the image processing section 206 executes OCT analysis processing, and at step S50 the display control section 208 outputs analysis data of the analysis result, and ends the present processing routine. Note that the image processing section 206 may execute step S40 after step S30, or execute the processing of step S30 after step S40. In such cases, at step S50, the display control section 208 may output analysis data of analysis results for both fluoroscopic fundus contrast imaging analysis and OCT analysis processing, and end the present processing routine.

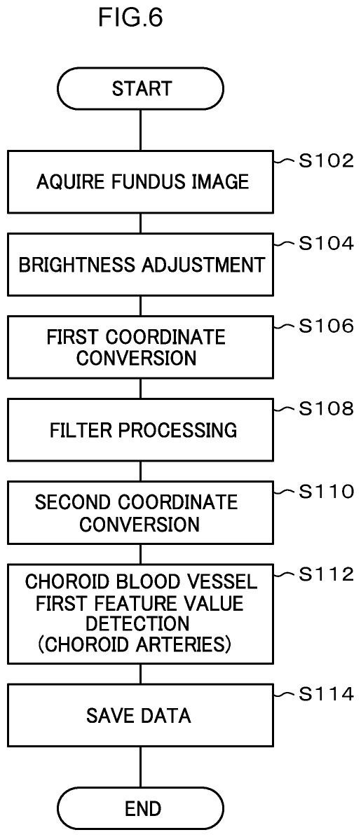

Next, description follows regarding fluoroscopic fundus contrast imaging analysis processing, with reference to FIG. 6.

The fluoroscopic fundus contrast imaging analysis processing is processing to analyze a condition of choroid blood vessels including choroid arteries using a contrast dye such as Indocyanine Green.

At step S102, the image processing section 206 acquires a fundus image. Specifically, a fundus image at an early stage after administering the contrast dye is acquired from the storage device 254. Namely, this fundus image is acquired, as an early-stage fundus image, by a fundus image (for example an SLO image) from out of fundus images imaged in a time series that was imaged after a predetermined specific time period had elapsed from when the contrast dye was administered, and that was imaged within a specific time period range up to when a separately predetermined specific time period has elapsed. The specific time period range may employ a time period range obtained experimentally as an initial time when the contrast dye has flowed through the choroid blood vessels, or may employ a time period range pre-set to when the contrast dye is predicted to flow through the choroid blood vessels. Moreover, plural images that were imaged in a time series may be classified into a front half of early images and a latter half of later images, and an early-stage fundus image may be selected from out of the images classified as early images.

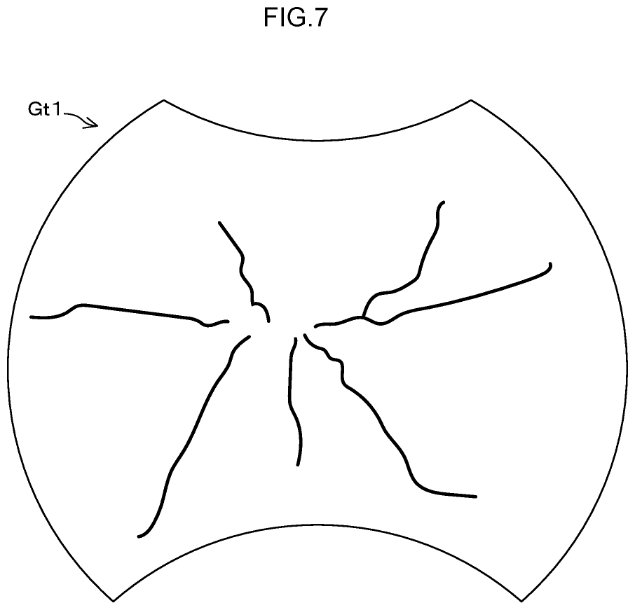

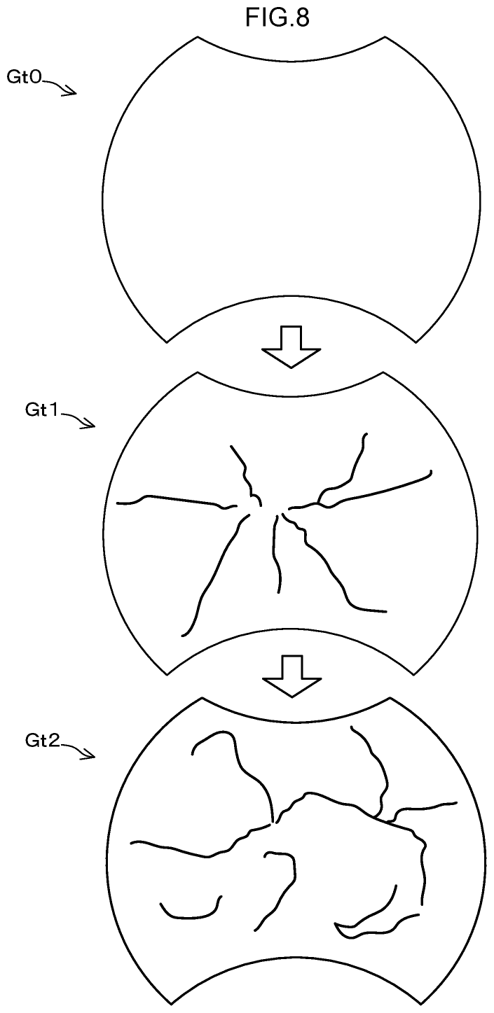

FIG. 7 is a diagram illustrating an early-stage fluoroscopic UWF-SLO image imaging florescent light, as an example of a fundus image of choroid blood vessels including choroid arteries. FIG. 8 is an example of fundus images illustrating a time series of fundus images around the time of contrast imaging. As illustrated in FIG. 8, choroid blood vessels do not appear in the fundus image Gt0 of a state prior to administering the contrast dye (before contrast imaging). However, in a fundus image Gt1 of a state in which the contrast dye has started to flow through the choroid blood vessels (at an early stage), most of the contrast dye flows in the choroid arteries, and florescent light appears mainly in the choroid arteries. Then in a fundus image Gt2 of a state in which the contrast dye has filled the choroid blood vessels (at a late stage), florescent light appears in substantially all blood vessels including the arteries and veins of the choroid.

Next, at step S104 of FIG. 6, the image processing section 206 performs brightness adjustment. This brightness adjustment is processing to highlight the choroid blood vessels (for example, the choroid arteries) appearing in the fluoroscopic UWF-SLO image. The processing to highlight the choroid blood vessels is processing to increase a ratio between the brightness of the background image and the brightness of the blood vessel image, compared to the ratio before brightness adjustment and, for example, includes processing to normalize a luminosity difference between a maximum and a minimum of luminosity values to a specific luminosity range, processing to adjust contrast, and the like. Moreover, as an example, the processing may include adjustment to remove, as noise, a distribution of minimum luminosity values, to apply a bias luminosity value to the luminosity values, to apply a multiplier to the luminosity values using a predetermined coefficient, and the like. In other words, the processing of step S104 corresponds to adjusting luminosity values so as to increase the dynamic range of the choroid blood vessel image in the fundus image.

At step S106, the image processing section 206 performs a first coordinate conversion. The first coordinate conversion is a polar coordinate conversion to convert coordinates in a Cartesian coordinate system into coordinates in a polar coordinate system. The origin of the polar coordinate conversion may be set to a predetermined position, such a center of the fundus image, to a position instructed by an operator or the like, to a position determined from a fundus structural object, such as an intermediate position between the macular and the optic nerve head or the like, and may be determined as the position of maximum luminosity values in the fundus image.

At step S108, the image processing section 206 performs filter processing on the fundus image that has been subjected to polar coordinate conversion, serving as the first coordinate conversion. The filter processing may be application of an image filter to highlight an image where brightness is contiguous in a specific direction, and Gabor Filter processing is applied in the present exemplary embodiment.

The choroid arteries extend in a radial pattern toward the periphery from a specific position. This means that the choroid arteries may be considered as containing line-shaped images having a contiguous brightness in a specific direction toward the periphery from a specific position at the center. Polar coordinate conversion is performed on the fundus image, and an image in which the brightness is contiguous in specific directions is extracted using filter processing. This thereby enables the choroid arteries to be highlighted.

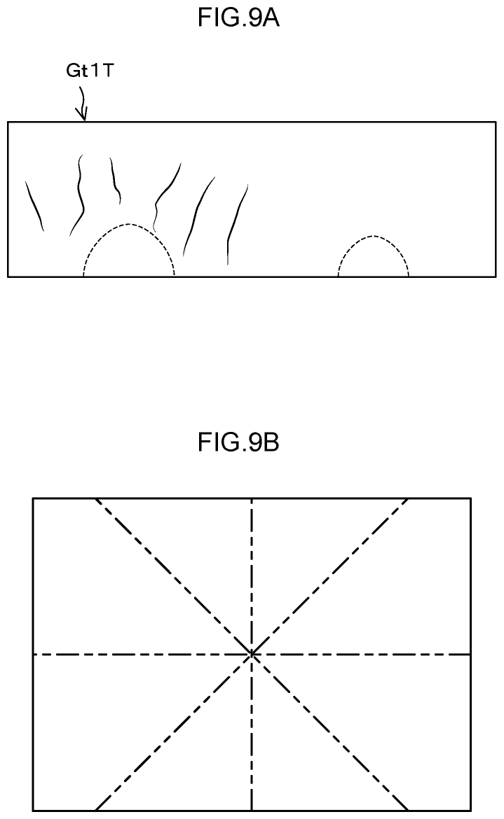

FIG. 9A is an example of an image resulting from subjecting a fundus image imaged by the ophthalmic device 110 to polar coordinate conversion and filter processing. As illustrated in FIG. 9A, blood vessels extending in specific directions corresponding to the choroid arteries are highlighted.

Next, at step S110 of FIG. 6, the image processing section 206 performs a second coordinate conversion. The second coordinate conversion is Cartesian coordinate conversion to convert polar coordinate system coordinates into Cartesian coordinate system coordinates. This Cartesian coordinate conversion results in an image in which blood vessels extending in specific directions corresponding to the choroid arteries are highlighted, and is backwards conversion from a polar coordinate system into a Cartesian coordinate system image.

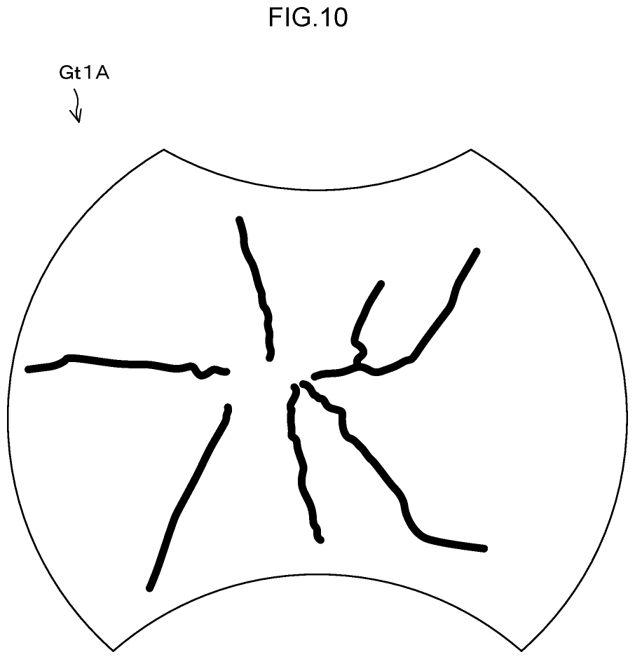

FIG. 10 is a diagram illustrating a fundus image Gt1A serving as an example of an image that has been backwards converted from a polar coordinate system image into a Cartesian coordinate system image. As illustrated in FIG. 10, images extending in specific directions corresponding to choroid arteries are highlighted compared to the drawing illustrated in FIG. 7. Namely, an image is formed in which the choroid arteries are distinct in the early-stage fundus image after contrast dye administration.

Next, at step S112 of FIG. 6, the image processing section 206 performs detection of a first feature value of the choroid blood vessels. The first feature value of the choroid blood vessels is information indicating an extent to which images of choroid arteries appear in the early-stage fundus image after contrast dye administration. For example, a choroid artery image is detected in the fundus image backwards converted into a Cartesian coordinate system image. The detection of the choroid artery image may be performed by counting pixels exceeding a predetermined luminosity value. A proportion of the pixels configuring the detected choroid artery image to the pixels of the fundus image as a whole is taken as the first feature value. This first feature value enables quantification of the condition of the choroid arteries appearing in the early-stage fundus image after contrast dye administration.

At step S114, the image processing section 206 saves data including the above fundus image and the first feature value. Specifically, image data representing the fundus image backwards converted at step S110, and data representing the first feature value detected at step S112, is saved in the RAM 266 or the storage device 254, and the present processing routine is ended.

Although a case has been described above in which processing has been executed on the imaged fundus image for polar coordinate conversion, filter processing, and Cartesian coordinate conversion, part of the fundus image that was imaged may be set as the image processing subject, and the above processing executed thereon. For example, in the fundus image that has been imaged, an image contained in a region surrounded by curves or straight lines in the shape of a circle, an ellipse, or a polygon of predetermined size, or a size specified by an operator, may be extracted as the image processing subject. Moreover, the above fundus image may contain a predetermined structural object present on the fundus, such as the macular, the optic nerve head, or the like.

The above saved data (step S114) is output as analysis data by the display control section 208.

Next, description follows regarding output of analysis data using the saved data. The analysis data is contained in a display screen to display an image (2D image) related to choroid blood vessels including choroid arteries. The display screen is generated by the display control section 208 of the server 140 based on instruction from the user, and is output as an image signal to the viewer 150. The viewer 150 displays the display screen on a display based on this image signal.

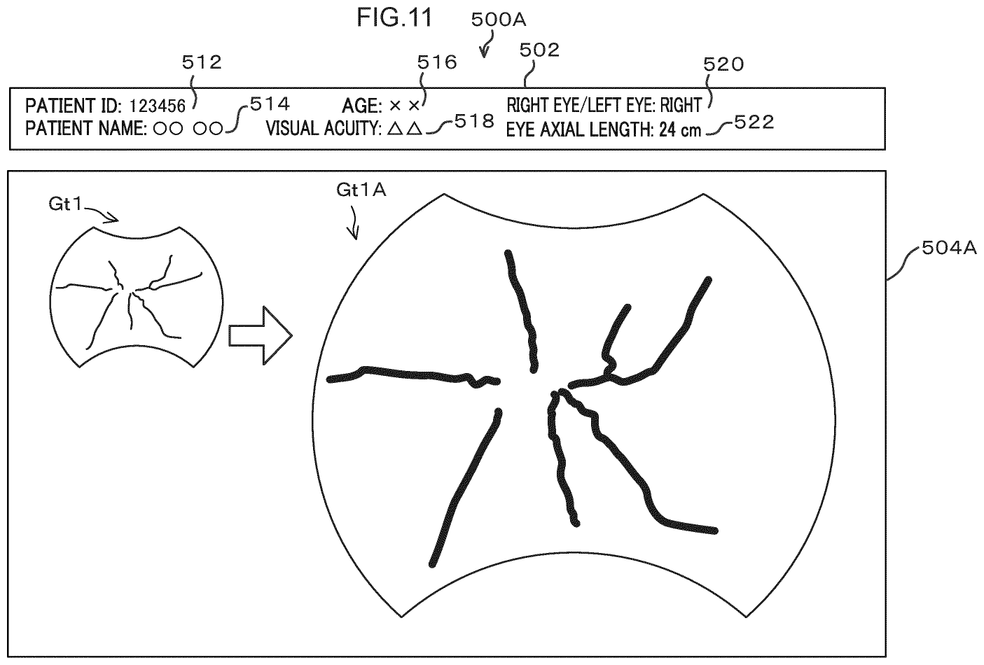

FIG. 11 illustrates a display screen 500A. As illustrated in FIG. 11, the display screen 500A includes an information area 502 and an image display area 504A.

The information area 502 includes a patient ID display field 512, a patient name display field 514, an age display field 516, a visual acuity display field 518, a right eye/left eye display field 520, and an eye axial length display field 522. Based on the information received from the server 140, the viewer 150 displays various information in each of the respective display regions from the patient ID display field 512 to the eye axial length display field 522.

The image display area 504A is a region to mainly display an examined eye image and the like. Display fields are set in the image display area 504A to display the fundus image Gt1 of a state (early stage) in which the contrast dye has started to flow through the choroid blood vessels, as described above, and the fundus image Gt1A formed as an image in which the choroid arteries are distinct. Note that, although omitted from illustration, fields may also be provided in the image display area 504A to display patient treatment history, or to function as a comment column for free input of a result observed by an ophthalmologist, i.e. the operator, and/or a diagnostic result.

FIG. 11 illustrates the fundus image Gt1 at an early stage of contrast imaging as an UWF-SLO image, and the fundus image Gt1A that has been subjected to image processing to make the choroid arteries distinct, contained in the display screen 500A as an image to indicate analysis results.

As described above, by executing image processing including fluoroscopic fundus contrast imaging analysis processing, the choroid arteries are extracted based on an image illustrating the choroid blood vessels to generate an image in which the choroid arteries are highlighted, enabling the choroid arteries to be visualized on the choroid imaged by fluoroscopic fundus contrast imaging.

Moreover, as described above, the first feature value of choroid blood vessels is detected, enabling the condition of the choroid arteries appearing in an early-stage fundus image after contrast dye administration to be quantified using the first feature value.

Although a case has been described above in which the first coordinate conversion is performed at step S104 on the fundus image and then, after filter processing has been performed at step S108, the second coordinate conversion is performed at step S110, the fluoroscopic fundus contrast imaging analysis may omit the first coordinate conversion of step S106 and the second coordinate conversion of step S110.

Description follows regarding the processing of step S108 for a case in which the processing of step S106 and step S110 has been omitted. FIG. 9B is a schematic diagram illustrating setting regions in a fundus image imaged by the ophthalmic device 110 for each specific center angle about a specific position at the center. The image processing section 206 sets regions on the fundus image for each specific center angle about the specific position as illustrated in FIG. 9B, and performs filter processing to highlight in each region images where brightness is contiguous in specific directions. The filter processing is performed in a target region for each of plural predetermined specific directions toward the periphery from the specific center position of the fundus image. The plural obtained images having different highlight directions are then combined at a specific combination ratio, thereby enabling, for the target region, an image to be extracted with contiguous brightness in directions from the specific center position of the fundus image toward the periphery. Performing such filter processing for all of the set regions enables a fundus image to be acquired in which choroid arteries spreading out in a radial pattern from the specific position are highlighted, without polar coordinate conversion being performed.

Moreover, a case has been described above in which the fluoroscopic fundus contrast imaging analysis processing is performed on a fluoroscopic fundus image generated using a contrast dye. The technology disclosed herein is not limited to performing fluoroscopic fundus contrast imaging analysis processing on a fluoroscopic fundus image generated using a contrast dye. For example, the fluoroscopic fundus contrast imaging analysis processing may be performed on an image in which blood vessels have been visualized without employing a contrast dye, such as an OCTA image or the like imaged by OCT angiography.

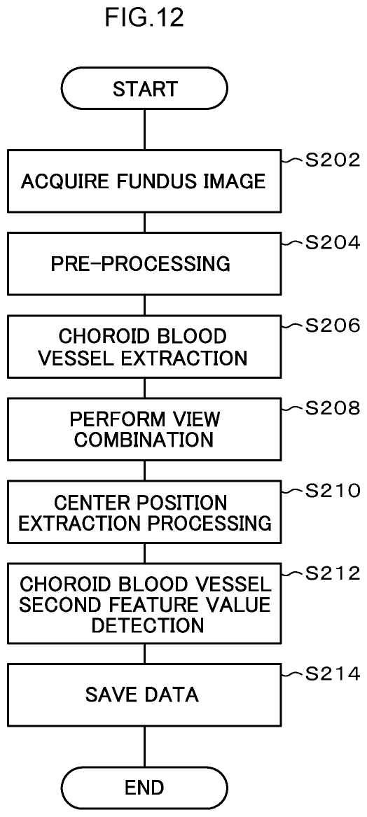

Next, description follows regarding OCT analysis processing, with reference to FIG. 12.

OCT analysis processing is processing using an OCT image to analyze the condition of choroid blood vessels including choroid arteries.

At step S202, the image processing section 206 acquires, from the storage device 254, OCT volume data corresponding to a fundus image including the choroid. Namely, after pre-processing such as blur processing to remove noise components has been executed at step S204, the image processing section 206 executes choroid blood vessel extraction processing at step S206. The effect of speckle noise is excluded by the blur processing, and Gaussian blur processing or the like is applicable therefor.

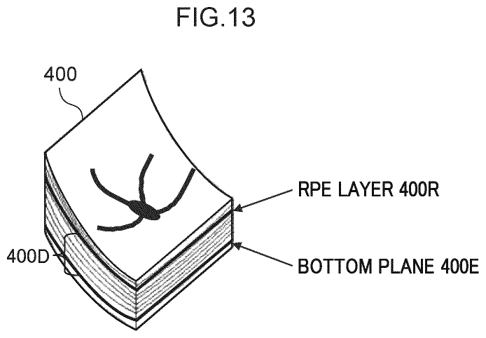

Specifically, as illustrated in FIG. 13, OCT volume data 400 is obtained by OCT imaging an examined eye using the ophthalmic device 110, with the OCT volume data 400 being a region with a specific surface area, for example a 6 mm×6 mm rectangle. Plural planes having different depths are set in the OCT volume data 400. A region where the choroid blood vessels are predicted to be present is extracted from the OCT volume data 400. This extraction processing enables a plane (bottom plane 400E) of a region deeper than the retinal pigment epithelium layer (hereafter referred to as the RPE layer) (a region further away than the RPE layer when looking from the eyeball center) to be extracted as a choroid region from a plane a specific number of pixels below, for example 10 pixels below, the RPE layer.

The image processing section 206 then removes noise components (pre-processing) and generates plural en-face images corresponding to each of the plural planes set therein. Each of the en-face images respectively generated to correspond to each plane is saved by the image processing section 206 in the RAM 266. The image processing section 206 thereby generates and saves en-face images. The en-face images may be generated from pixel values of pixels present in the corresponding planes, and a pixel group in the shallow direction including the corresponding plane, and a pixel group in the deep direction, may be extracted from the OCT volume data 400, and pixel values derived may be the mean or median of the luminosity values of these pixel groups. Image processing may be employed to remove noise or the like when finding the pixel values.

Instead of using a plane 10 pixels below the RPE layer as a reference, as described above, a plane 10 pixels below the Bruch's membrane, which is present directly below the RPE layer, may, for example, be employed therefor. Note that 10 pixels below in the A-scan direction when OCT volume data was generated may be employed to identify a position 10 pixels below. The number of pixels to determine the plane is not limited to 10 pixels, and a freely selected number of pixels may be set. Moreover, instead of by number of pixels, definition may be by length in millimeters, microns, or the like.

The choroid blood vessel extraction processing may employ any line-extraction processing capable of extracting blood vessels that reflects the shape of the blood vessels. The image processing section 206 accordingly extracts the choroid blood vessels from the OCT volume data 400D by executing line-extraction processing on the OCT volume data 400D that has been subjected to pre-processing. Specifically, the image processing section 206 performs image processing using, for example, an eigenvalue filter, a Gabor filter, or the like to extract line shaped blood vessel regions from the OCT volume data 400D. The blood vessel regions in the OCT volume data 400D are pixels of low luminosity (blackish pixels), and regions where low luminosity pixels are contiguous remain as blood vessels portions.

At step S208, the image processing section 206 performs view combination to combine the plural extracted images of the choroid results to derive an image visualizing a larger region than regions obtained at each time of OCT imaging, and saves this image in the RAM 266. Specifically, the above processing is executed on regions of interest obtained by OCT imaging having different fields of view, namely for each of plural different regions, and the images obtained thereby are combined.

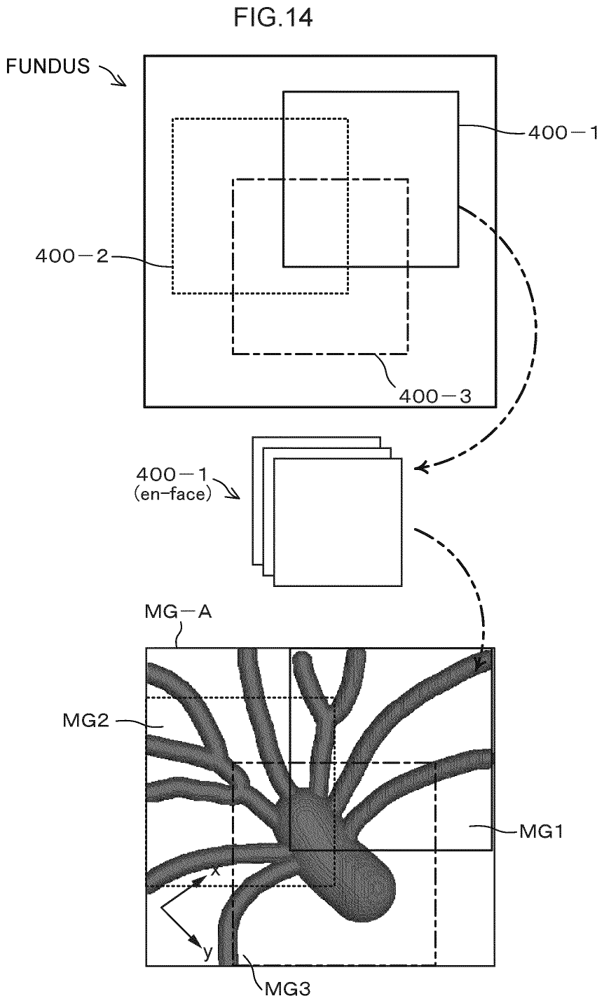

FIG. 14 schematically illustrates a procedure up to choroid blood vessel extraction. In the example illustrated in FIG. 14, in order to make the region where the choroid blood vessels are visualized larger than the regions obtained at each time of OCT imaging (rectangular regions of a specific surface area), plural (three) regions that have at least a mutually overlapping portion are employed. Respective OCT volume data 400-1, 400-2, 400-3 obtained by OCT imaging of each of the plural (three) regions is acquired from the storage device 254, and after pre-processing (step S204), the choroid blood vessels are extracted (step S206). Choroid blood vessel images MG1, MG2, MG3 are then combined to obtain a choroid blood vessel image MG-A. Note that en-face images generated from each of the choroid blood vessel images MG1, MG2, MG3 may be employed for combination. The choroid blood vessels are joined in the choroid blood vessel image MG-A due to en-face images equivalent to each other in depth direction being combined. The choroid blood vessels obtained by OCT imaging and extraction from plural different regions are combined in three dimensions in this manner, enabling the choroid blood vessels to be visualized for a larger range than the range of the choroid blood vessels obtained by independent OCT imaging. Note that as long as the range that an operator wishes to observe is contained in the range of choroid blood vessels obtained by independent OCT imaging, step S208 may be skipped, such that step S210 is executed as the next step to step S206.

Next, at step S210 of FIG. 12, the image processing section 206 executes choroid blood vessels center position extraction processing. The choroid blood vessel center position extraction processing is processing to derive center lines passing through the centers of the choroid blood vessels. In the present exemplary embodiment, the blood vessel center lines are representative lines indicating extension directions of the blood vessels. Note that the blood vessel center lines may be lines passing through positions slightly displaced from the center of the choroid blood vessels as long the extension directions of the blood vessels can be ascertained. Note that the center position extraction processing is processing sometimes called skeletonization (framework generation) processing or image line thinning processing, and indicates processing to make a line drawing image.

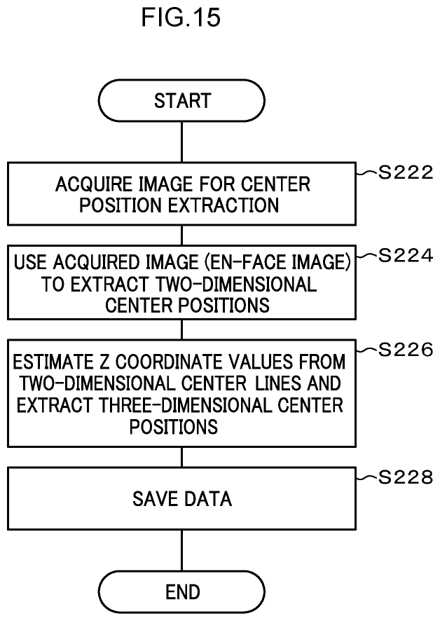

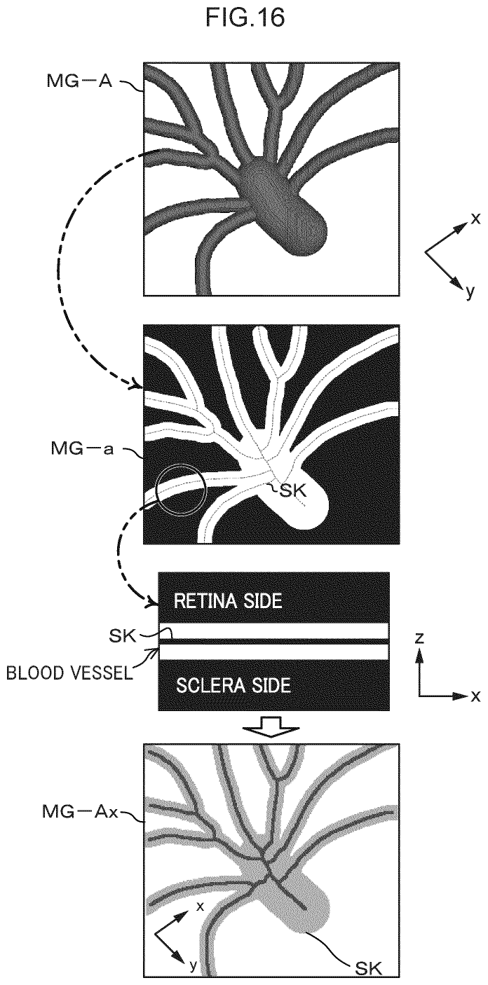

Description follows regarding the choroid blood vessel center position extraction processing, with reference to FIG. 15 and FIG. 16. In the present exemplary embodiment, center lines expressed in two dimensions for the choroid blood vessels are derived from the en-face images, and center lines expressed in three dimensions are derived from the information of the plural extracted two-dimensional center lines.

At step S222 illustrated in FIG. 15, the image processing section 206 acquires an image for center position extraction. A two-dimensional image including a choroid blood vessel image may be employed as the image for center position extraction. Specifically, a single en-face image may be extracted from out of en-face images generated from the OCT volume data 400, or a two-dimensional image generated by performing image processing on plural en-face images may be employed. For example, as illustrated in FIG. 16, a single en-face image containing the choroid blood vessel image MG-A is acquired as an image MG-a for center position extraction.

At step S224, the image processing section 206 executes center position extraction processing to derive two-dimensional center lines using the image acquired at step S222. Specifically, line segments indicating the two-dimensional center lines are derived from the acquired two-dimensional image (for example, a single en-face image). An example of two-dimensional center position extraction processing that may be employed is application of processing in which an image indicating a choroid blood vessel region is repeatedly dilated and erroded until becoming a line segment, with the line segments finally obtained (for example, a pixel group of single pixels contiguous in the blood vessel direction) taken as two-dimensional center lines SK. For example, for the image MG-a that is a single en-face image, as illustrated in FIG. 16, representative lines of the extension of the choroid blood vessels (indicated by dotted lines in FIG. 16) are derived as the choroid blood vessel center lines SK.

At step S226, the image processing section 206 executes center position extraction processing in three dimensions by estimating depth direction positions (Z coordinate values) from the two-dimensional center lines SK derived at step S224. Specifically, the image processing section 206 appends depth direction Z coordinate values to the center lines SK represented in two dimensions to derive three-dimensional center lines SK. For example, the choroid blood vessels including the two-dimensional center lines SK are positioned at depths that correspond to the extraction positions of respective en-face images. When doing so, assuming that the blood vessels have a circular tube shape, then the cross-section of a three-dimensional center line SK is a cross-section positioned evenly on the retina side and the sclera side. The extraction position of the en-face image is accordingly appended as an estimated value of the Z coordinate values to derive the three-dimensional center lines SK.

Note that such Z coordinate value estimation processing may be performed by applying morphological processing to data generated by acquiring the choroid blood vessels in the depth direction (Z direction) in a freely selected XY plane along the center line, namely, to data indicating a cross-section profile of the choroid blood vessels. Moreover, such Z coordinate value estimation processing may employ graph shortest path search processing. Graph shortest path search processing is processing to estimate the Z coordinate values of the center lines by acquiring, from out of a freely selected XY plane along a center line, choroid blood vessels only at branch points, where the extracted center lines branch into plural center lines, and center line end points, to estimate the Z coordinates, and by connecting these estimated Z coordinate values together with the shortest distance.

At step S228, the image processing section 206 saves, in the RAM 266 or the storage device 254, data indicating the three-dimensional center lines derived at step S226 and then ends processing. FIG. 16 illustrates a schematic image in which the three-dimensional center lines SK derived as described above are displayed superimposed on the choroid blood vessel image MG-A as a choroid blood vessel image MG-Ax.

A case has been described above in which the three-dimensional center lines extracted from a single en-face image are derived (Z coordinate values are estimated) from out of the en-face images generated from the OCT volume data 400. The technology disclosed herein is not limited extracting a single en-face image for use. For example, an image generated by combining plural en-face images may be applied as the single en-face image.

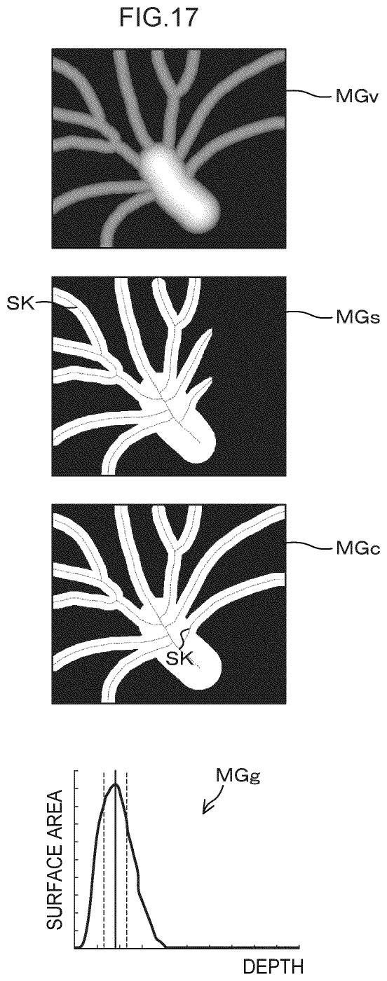

FIG. 17 is a schematic diagram related to an image combining plural en-face images applied as the single en-face image. FIG. 17 illustrates a schematic image MGv resulting from taking the mean brightness of plural en-face images generated from the OCT volume data 400. Moreover, a single en-face image MGs extracted from out of plural en-face images is illustrated. A combined image MGc generated by combining plural en-face images is illustrated. A relationship between depth of plural en-face images and surface area occupied by choroid blood vessels is illustrated as a graph image MGg.

As illustrated in FIG. 17, compared to the schematic image MGv, missing portions of choroid blood vessels are present in the extracted single en-face image MGs. There is accordingly a reduction in the precision of the center lines SK obtained. This is because the choroid blood vessels extend while meandering with respect to the depth direction (Z direction). For example, consider a case in which, with respect to the Z direction, a choroid blood vessel extends toward the +Z direction (a case of extending in an upward orientation, a case of extending toward the eyeball center), then only a part of the blood vessel is extracted in the en-face image at an identified depth position. Choroid blood vessels are blood vessels that have been extracted from en-face images at positions shallower than the identified depth position. There are accordingly choroid blood vessel missing portions present when considering only a single en-face image MGs. To address this issue, the combined image MGc resulting from combining plural en-face images is accordingly applied as the single en-face image. The combined image MGc may, as illustrated by the graph image MGg, employ only en-face images for which the surface area of the choroid blood vessels exceeds a specific value (for example, up to a specific number of en-face images away from the maximum surface area). Employing an image combining plural en-face images as the single en-face image in this manner enables the center lines SK for each blood vessel includes the choroid blood vessel to be accurately represented, while estimating an extension direction of the choroid blood vessel with respect to the Z direction. Note that the combined image MGc is not necessarily generated from plural en-face images. The center position extraction processing is executed for each of the plural en-face images acquired at different depth direction positions. Information about the center lines extracted from each en-face image may be combined with Z coordinate values so as to derive three-dimensional center lines SK. Moreover, an en-face image at each depth position may be employed so as to find the circularity/ellipticity of blood vessel cross-sections. The circularity/ellipticity is found from depth information of en-face images and blood vessel occupied surface area in en-face images, with a circular profile indicated by the occupied surface area changing in proportion to the change in depth, and an elliptical profile indicated when the change in occupied surface area gets greater or smaller than the change in depth.

The image processing section 206 transitions processing to step S212 illustrated in FIG. 12 when the center position extraction processing described above has finished. At step S212, the image processing section 206 executes processing to detect a second feature value of the choroid blood vessels. The processing to detect the second feature value of the choroid blood vessels is processing to detect information indicating a feature related to a shape of the choroid blood vessels. In the present exemplary embodiment, as described later, a cross-sectional area and a blood vessel diameter of the choroid blood vessels are detected as an example of second feature values of the choroid blood vessels. Note that a fundus image that has not been subjected to the center position extraction processing may be employed in a step to detect the second feature value of the choroid blood vessels. In such cases, a configuration may be adopted so as to execute step S212 as the next step after the step S206, or so as to execute step S212 as the next step after the step S208.

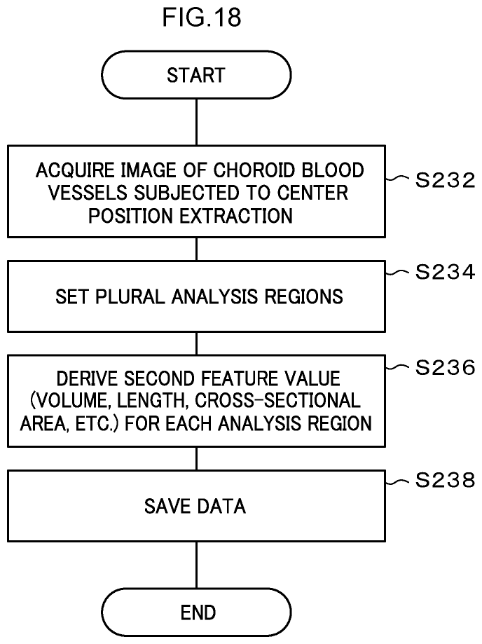

Description follows regarding processing to detect the second feature value of the choroid blood vessels, with reference to FIG. 18.

At step S232 illustrated in FIG. 18, the image processing section 206 acquires an image of the choroid blood vessels subjected to center position extraction (see image MG-Ax illustrated in FIG. 16). Next, at step S234, the image processing section 206 sets plural analysis regions on the acquired choroid blood vessel image.

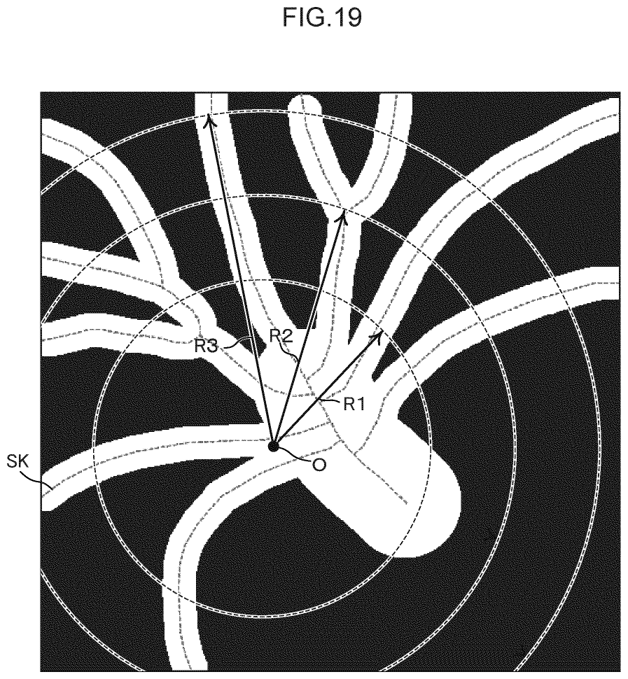

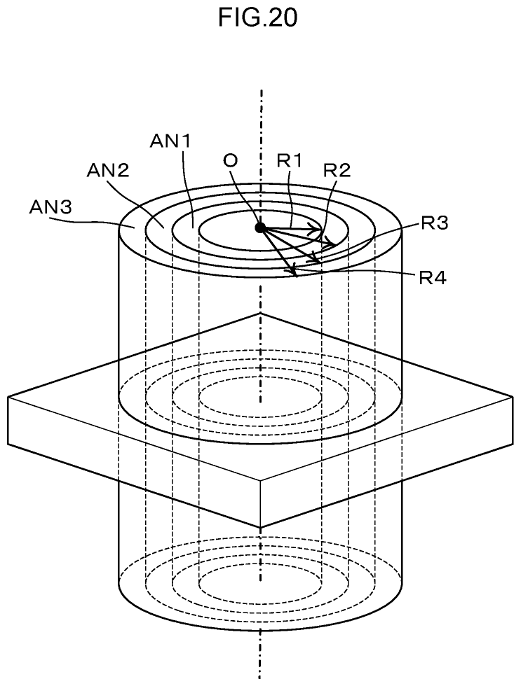

The analysis regions employ regions on the choroid region from a first region indicating a first cross-section at a position distanced by a first specific distance from a predetermined specific position, to a second region indicating a second cross-section at a position distanced from the specific position by a second specific distance different to the first specific distance. For example, the first cross-section and the second cross-section may employ as delineation sections of curved lines centered around a specific position on the bottom plane. As a specific example, as illustrated in FIG. 19 and FIG. 20, concentric circles centered around a specific position O may be taken as delineation, and the choroid region divided into concentric circle regions (circular pillar-shaped regions including the depth direction) delineated along the depth direction of the choroid may applied as the analysis regions. FIG. 19 is a diagram illustrating analysis regions on a plan view of the choroid region delineated by plural concentric circles centered on the specific position O. FIG. 20 illustrates a perspective view of analysis regions AN1, AN2, AN3 arising from a flat plate shaped choroid region being divided into circular pillar-shapes by delineating the bottom plane with plural concentric circles centered on the specific position O.

The predetermined specific position O may be set manually when setting the analysis region, such as by setting an instructed position as the site of interest while the operator is checking the choroid blood vessel images and the like. Moreover, for example, the analysis regions may be set so as to perpendicularly cross blood vessels extending from the bulge portion of the choroid blood vessels. A center of a predetermined structural object on the fundus, such as the center of the bulge portion or the like, or a predetermined position, may be set.

In the example illustrated in FIG. 20, the analysis regions are set as an analysis region AN1 arising from dividing the choroid region by delineating with circles of radii R1, R2 (>R1) centered on the specific position O, an analysis region AN2 delineated by circles of radii R2, R3 (>R2), and an analysis region AN3 delineated by circles of radii R3, R4 (>R3). In this manner, plural analysis regions are set from the specific position O toward the outside, or plural analysis regions are set from the outside toward the specific position O, in a direction intersecting with the depth direction of the choroid region. Note that the analysis regions AN1, AN2, AN3 may be set so as to overlap with a part of an adjacent analysis region, or may be set so as to be separated from each other by a predetermined separation.

Note that the analysis regions are not limited to being concentric circle regions. For example, the analysis regions may be elliptical with a center at a predetermined specific position, may be oval with a center at a specific position, or may be a circular arc with a center at a specific position. Namely, the analysis regions may be set on the image so as to be superimposed on the choroid blood vessels. Moreover, when forming the concentric circle regions, there is no limitation to setting the analysis regions to circular cylinder shapes. For example, the analysis regions may be regions that arise by separating by curved lines into plate shapes that configure parts of concentric spheres.

When the setting of the analysis region is finished, at step S236 illustrated in FIG. 18, the image processing section 206 derives the second feature value for each of the analysis regions set. In the present exemplary embodiment, the second feature values of the choroid blood vessels are detected by deriving the cross-sectional area and blood vessel diameter of the choroid blood vessels. Specifically, the image processing section 206 employes a volume of the choroid blood vessels, and a blood vessel length of the choroid blood vessels, to derive a mean blood vessel diameter and a mean cross-sectional area of the choroid blood vessels.

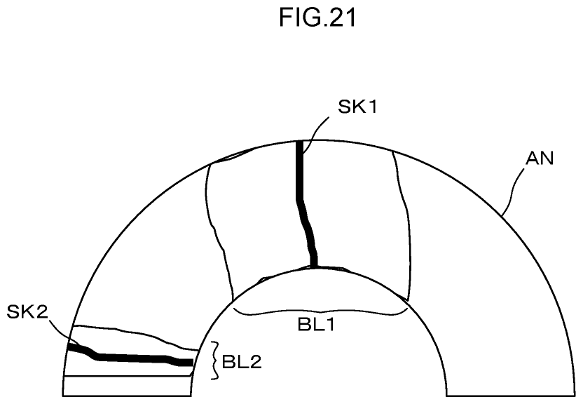

FIG. 21 illustrates a schematic diagram of an analysis region AN. In the example illustrated in FIG. 21, the analysis region AN includes a first blood vessel region BL1 including a first center line SK1, and a second blood vessel region BL2 including a second center line SK2. The number of pixels of the first blood vessel region BL1 is computed in the analysis region AN and taken as a volume V1 of the first blood vessel region BL1. Moreover, the number of pixels of the first center line SK1 is computed and taken as a blood vessel length L1 of the first blood vessel region BL1. Similarly, the number of pixels of the second blood vessel region BL2 is computed and taken as a volume V2 of the second blood vessel region BL2, and the number of pixels of the second center line SK2 is computed and taken as a blood vessel length L2 of the second blood vessel region BL2. A mean cross-sectional area Sa can be derived by dividing the total value (V) of the volume of the blood vessel region in the analysis region AN by the total value (L) of the blood vessel length.

$$Sa = ( V ) / ( L )$$

For example, (V)=V1+V2, (L)=L1+L2

The blood vessel diameter may be a radius. For example, the mean blood vessel diameter ra in the analysis region AN can be taken as corresponding to a radius rb of a circle having a common surface area to the derived mean cross-sectional area Sa under an assumption that the cross-section of the blood vessels is a circular shape.

$$Sa = π \cdot {( ra )}^{2}$$

$$Namely , {( ra )}^{2} = {( r b )}^{2} = Sa / π$$

Note that the mean blood vessel diameter ra in the analysis region AN may be derived using the circularity/ellipticity as described above.

The mean cross-sectional area Sa of the blood vessel region in the analysis region AN, and the mean blood vessel diameter ra corresponding to a circular shaped cross-section, are derived, for each of the analysis regions, as second feature values in the analysis region AN. The mean cross-sectional area Sa derived as the second feature value in the analysis region AN, and the mean blood vessel diameter ra, are examples of physical quantities related to the shape of the choroid blood vessels of the present disclosure. Examples of the second feature value include a mean blood vessel length, and a skeleton density (number of pixels of center lines with respect to a unit surface area in an image or with respect to a number of pixels of the entire image).

Next, at step S238, the image processing section 206 saves, either in the RAM 266 or the storage device 254, data indicating the mean cross-sectional area Sa and the mean blood vessel diameter ra as the second feature values of the analysis region derived at step S236, and ends the processing. Note that, as the second feature values, data representing the volume V1 and the blood vessel length L1 of the first blood vessel region BL1, and the volume V2 and the blood vessel length L2 of the second blood vessel region BL2, may be saved in the RAM 266 or the storage device 254. Note that at step S238, not only the second feature values, but also position information for the specific position O of the first blood vessel region BL1 and the second blood vessel region BL2 may be saved in the RAM 266 or the storage device 254.

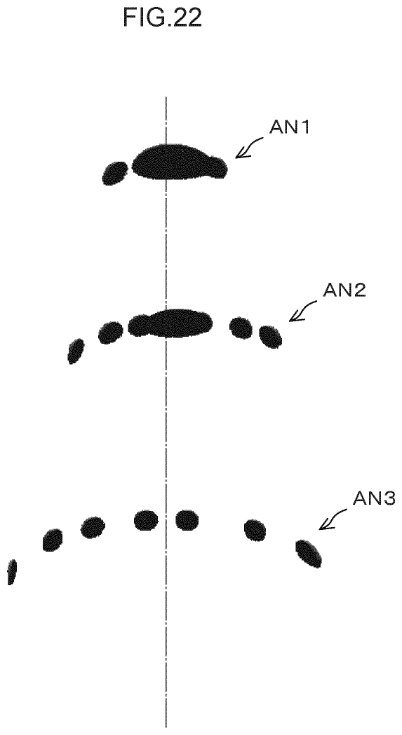

By setting the plural analysis regions described above to, for example, different regions of concentric circle regions having concentric circular shapes, as illustrated in FIG. 22, a condition (for example, a shape distribution or the like) of the choroid blood vessels in the choroid can be observed by the operator by displaying plural analysis regions in a specific sequence. Moreover, for example, from the mean cross-sectional area Sa and the mean blood vessel diameter ra, a condition of the choroid blood vessels getting thinner/thicker as a distance from the specific position O gets further away, and the branch position/number of branches of the choroid blood vessels, the join position/join number of the choroid blood vessels, and the meandering characteristics in the depth direction (Z direction) of the choroid blood vessel extension direction, can be detected.

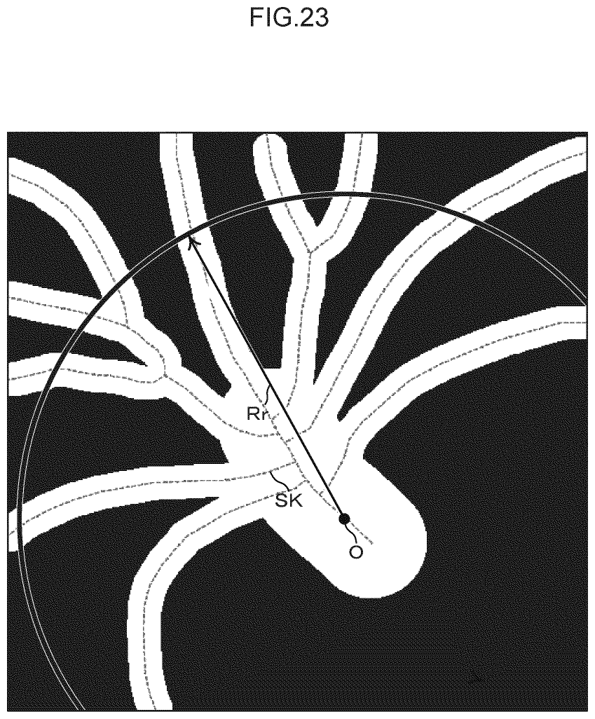

Note that although a description has been given above of a case in which plural analysis regions are set, technology disclosed herein is not limited thereto. For example, as illustrated in FIG. 23, a choroid region up to a predetermined radius Rr centered on the above predetermined specific position O may be employed as the analysis region.

The data saved above (step S214) is output by the display control section 208 as analysis data.

Next, description follows regarding output of the analysis data from the saved data. The analysis data includes a display screen to display analysis results by OCT analysis. This display screen is generated by the display control section 208 of the server 140 based on user instruction, and is output as an image signal to the viewer 150. The viewer 150 displays the display screen on a display based on this image signal.

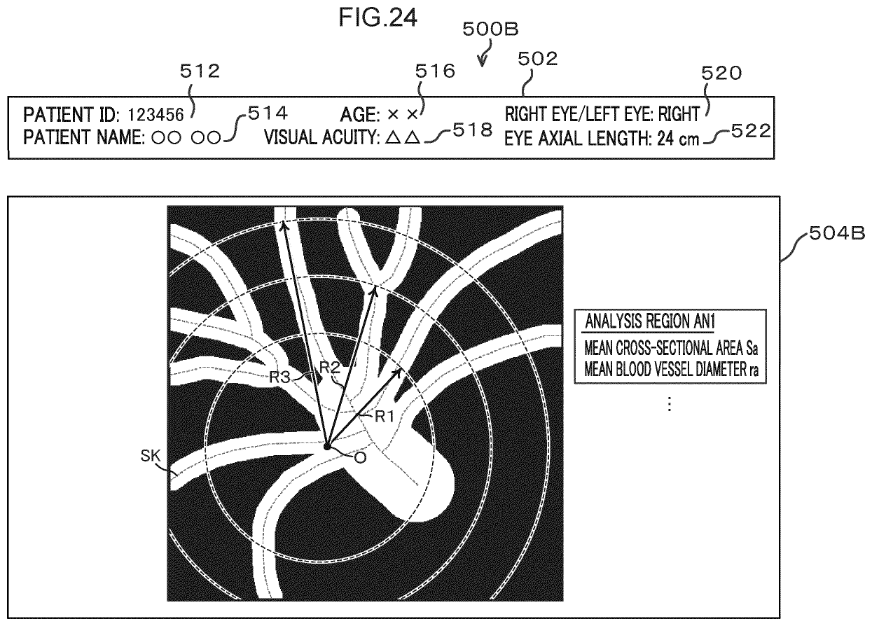

FIG. 24 illustrates a display screen 500B. As illustrated in FIG. 24, the display screen 500B includes an information area 502 similar to that of the display screen 500A, and an image display area 504B.

The image display area 504B is an area to display analysis results of the above OCT analysis processing and the like. A configuration may be adopted so as to include, in the image display area 504B, the above analysis results chart (FIG. 19), in which plural concentric circles centered on the specific position O are delineated on a plan view of the choroid region.

Another example of an output of the analysis data described above may be applied to a modified example in which various types of visualization display images are included in the display screen.

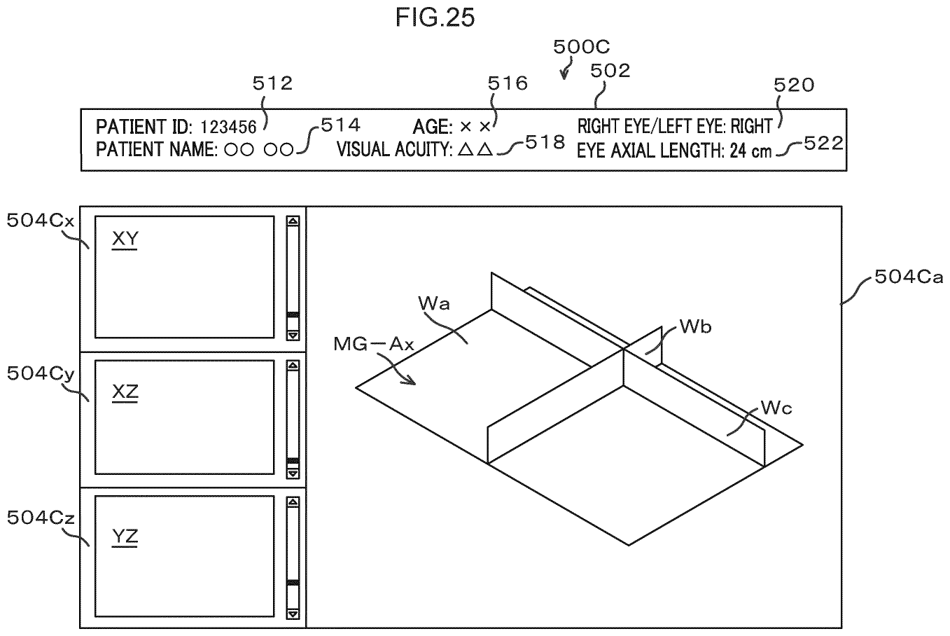

In a first modified example, as illustrated in FIG. 25, cross-sections at freely selected positions can be employed as analysis region diagrams for the choroid blood vessel image of the analysis results (for example, the image MG-Ax illustrated in FIG. 16).

A display screen 500C of the first modified example, includes an information area 502 similar to that of the display screen 500A, and image display areas 504Ca, 504Cx, 504Cy, 504Cz.

The image display area 504Ca is a region to display the choroid blood vessel image MG-Ax (FIG. 16) as the analysis result of above OCT analysis processing. The image display area 504Ca includes a moveable frame plane Wa for presenting the choroid blood vessels as a cross-section in an XY plane at a freely selected Z coordinate value. An image display area 504Cx is a region to display an image MG-Ax of the choroid blood vessels as a cross-section in an XY plane at a freely selected Z coordinate value interlocked to movement on the frame plane Wa. Similarly, the image display area 504Ca includes a moveable frame plane Wb for presenting the choroid blood vessels as a cross-section in an XZ plane at a freely selected Y coordinate value. The image display area 504Cy is a region to display an image MG-Ax of the choroid blood vessels at a cross-section in an XZ plane at a freely selected Y coordinate value, interlocked to movement of the frame plane Wb. Moreover, a moveable frame plane Wc is included for presenting the choroid blood vessels as a cross-section in a YZ plane at a freely selected X coordinate value. The image display area 504Cz is a region to display an image MG-Ax of the choroid blood vessels as a cross-section in a YZ plane at a freely selected X coordinate value, interlocked to movement of the frame plane Wc.

In the first modified example, cross-sections at freely selected positions on a choroid blood vessel image can be visualized and presented in this manner, enabling an operator to check an image of the choroid blood vessels on the choroid at a freely selected position.

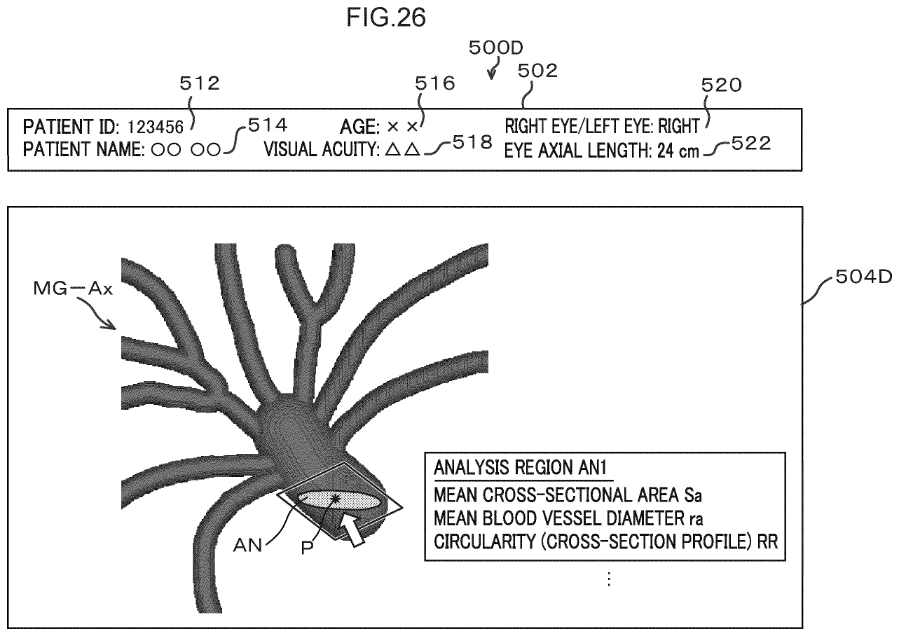

In a second modified example, as illustrated in FIG. 26, information indicating a choroid blood vessel analysis result at a freely selected position can be employed interlocked to a diagram.

A display screen 500D of the second modified example includes an information area 502 similar to that of the display screen 500A, and an image display area 504D.

The image display area 504D is a region to display an image MG-Ax of the choroid blood vessels (see FIG. 16) as analysis results or the like of the above OCT analysis processing. A configuration is adopted such that when an instruction of a freely selected position P on the choroid blood vessel image MG-Ax is received by the image processing section 206, based on an analysis region AN, information related to a cross-section of the choroid blood vessels at the position P is displayed.

Thus in the second modified example, information related to a cross-section of blood vessels at a freely selected position can be presented, while also presenting a choroid blood vessel image, enabling an operator to check information related to the cross-section of the choroid blood vessels in relation to an instructed position, which is a freely selected position.

In a third modified example, a display mode of a choroid blood vessel image MG-Ax (FIG. 16) of choroid blood vessel analysis results can be changed and then employed (omitted in the drawings). For example, a choroid blood vessel image is presented in a display mode, such as a display mode in which the color of layers is changed separately according to depth direction position before display. Moreover, a choroid blood vessel image is presented in a display mode, such as a display mode in which expansion or contraction is performed in at least one instructed direction from out of the XYZ axes before display. In this manner, due to enabling the display mode of the choroid blood vessel image to be changed, application can be made so as to present, as required, a location an operator wishes to pay attention to, or does not wish to pay attention to.

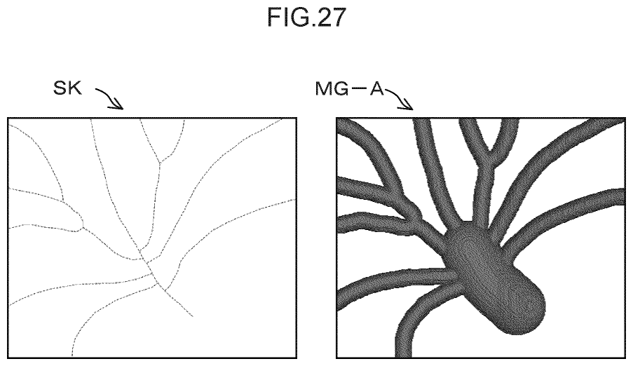

In a fourth modified example, as illustrated in FIG. 27, application can be made to separate presentation of a choroid blood vessel image and center lines. Separating the choroid blood vessel image and the center lines enables detection of center line branches, namely, divided blood vessels. Changing the display mode such as by changing the color or the like for each divided blood vessel enables an operator to check choroid blood vessels separated out in the choroid.

As described above, an image indicating a condition of the choroid blood vessels is presented by executing image processing including OCT analysis processing, enabling the choroid blood vessels to be visualized in various modes for the choroid imaged by OCT.

In the exemplary embodiment described above, although the image processing is executed by the server 140, the present disclosure is not limited thereto, and the image processing may be executed by the ophthalmic device 110, the viewer 150, or by an additional image processing device additionally provided on the network 130.

In the present disclosure, each of the configuration elements (devices and the like) may be present singly or present as two or more thereof as long as inconsistencies do not result therefrom.

In each of the examples described above respective examples are given of cases in which the image processing is implemented by a software configuration utilizing a computer, the present disclosure is not limited thereto, and at least part of the processing may be implemented by a hardware configuration. Although in the description above a CPU has been employed as an example of a general purpose processor, the term processor indicates a widely defined processor, and examples thereof include general purpose processors (for example, a central processing unit (CPU) or the like), and specialized processors (for example, a graphics processing unit (GPU), an application specific integrated circuit (ASIC), a field programmable gate array (FPGA), a programmable logic device, or the like). Thus the image processing may be executed by a hardware configuration alone, or part of the processing in the image processing may be executed by a software configuration and the remaining processing thereof may be executed by a hardware configuration.

Moreover, the above operations of the processor are not limited to being performed by a single processor, may be performed by plural processors collaborating with each other, and may be performed by cooperation between plural processors present at physically separated locations.

In order to execute the above processing using a computer, a program written with computer executable code for the above processing may be stored and distributed on a storage medium such as an optical disc or the like.

Although the technology disclosed herein has been described by way of exemplary embodiments, the above image processing is merely an example, and the scope of technology disclosed herein is not limited to the range of the above exemplary embodiments. Thus various modifications and improvements may be made to the above exemplary embodiments, such as redundant processing being omitted, new processing being added, and the processing sequence being swapped around within a range not departing from the spirit of the present disclosure, and these modified or improved embodiments are contained within the range of technology disclosed herein.

All publications, patent applications and technical standards mentioned in the present specification are incorporated by reference in the present specification to the same extent as if each individual publication, patent application, or technical standard was specifically and individually indicated to be incorporated by reference. Moreover, the entire content of the disclosure of Japanese Patent Application No. 2023-064527 filed on Apr. 11, 2023 is incorporated by reference in the present specification.

1. An image processing method performed by a processor, the image processing method comprising:
acquiring a first fundus image in which choroid blood vessels are visualized;
extracting choroid arteries in the first fundus image by performing image processing on the first fundus image; and
generating a second fundus image in which the choroid arteries are highlighted,
wherein,
extracting the choroid arteries includes
converting the first fundus image to a polar coordinate display image by polar coordinate conversion, and
processing to highlight an image, contained in the polar coordinate display image, in which brightness is continuous in a predetermined direction from a specific position toward a periphery; and
generating the second fundus image includes
converting the polar coordinate display image into a Cartesian coordinate display image by Cartesian image conversion.

2. (canceled)

3. An image processing method performed by a processor, the image processing method comprising:
acquiring a first fundus image in which choroid blood vessels are visualized;
extracting choroid arteries in the first fundus image by performing image processing on the first fundus image; and
generating a second fundus image in which the choroid arteries are highlighted,
wherein:
extracting the choroid arteries includes
setting a plurality of regions in the first fundus image for each specific center angle centered on a specific position, and
performing highlight processing in a predetermined plurality of directions to highlight images where brightness is continuous in a direction from the specific position toward a periphery in images contained in the set plurality of regions; and
generating the second fundus image step includes
combining, at a specific combination ratio, a plurality of highlight images in each of the regions obtained in the highlight processing to generate the second fundus image in which choroid arteries are highlighted.

4. The image processing method of claim 1, wherein:
the highlight processing is Gabor filter processing.

5. The image processing method of claim 1, wherein:
the first fundus image is a fluoroscopic fundus image imaged using a contrast dye.

6. The image processing method of claim 5, wherein the fluoroscopic fundus image is a fundus image imaged from after contrast dye application to an examined eye until a specific time elapses.

7. The image processing method of claim 1, wherein the first fundus image is an OCTA image imaged by OCT angiography.

8. The image processing method of claim 1, further comprising, based on extracted choroid arteries, deriving a feature value that gets larger with increasing brightness of an image representing the choroid arteries contained in the first fundus image.

9. The image processing method of claim 1, further comprising outputting data of processing results from any one of acquiring the first fundus image, extracting the choroid arteries or generating the second fundus image.

10. An image processing device, comprising:
an acquisition unit configured to acquire a first fundus image in which choroid blood vessels are visualized;
an extraction unit configured to extract choroid arteries in the first fundus image acquired by the acquisition unit by performing image processing on the first fundus image; and
a generation unit configured to generate a second fundus image in which the choroid arteries extracted by the extraction unit are highlighted,
wherein,
the extraction unit is configured to
convert the first fundus image to a polar coordinate display image by polar coordinate conversion, and
process to highlight an image, contained in the polar coordinate display image, in which brightness is continuous in a predetermined direction from a specific position toward a periphery; and
the generation unit is configured to
convert the polar coordinate display image subjected to highlight processing into a Cartesian coordinate display image by Cartesian image conversion.

11. (canceled)

12. An image processing device, comprising:
an acquisition unit configured to acquire a first fundus image in which choroid blood vessels are visualized;
an extraction unit configured to extract choroid arteries in the first fundus image acquired by the acquisition unit by performing image processing on the first fundus image; and
a generation unit configured to generate a second fundus image in which the choroid arteries extracted by the extraction unit are highlighted,
wherein,
the extraction unit is configured to
set a plurality of regions in the first fundus image for each specific center angle centered on a specific position, and
perform highlight processing in a predetermined plurality of directions to highlight images where brightness is continuous in a direction from the specific position toward a periphery in images contained in the set plurality of regions; and
the generation unit is configured to
combine, at a specific combination ratio, a plurality of highlight images in each of the regions obtained in the highlight processing to generate the second image in which choroid arteries are highlighted.

13. A non-transitory storage medium storing a program for causing a computer to execute processing comprising:
acquiring a first fundus image in which choroid blood vessels are visualized;
extracting choroid arteries in the first fundus image by performing image processing on the first fundus image; and
generating a second fundus image in which the choroid arteries are highlighted,
wherein,
extracting the choroid arteries includes
converting the first fundus image to a polar coordinate display image by polar coordinate conversion, and
processing to highlight an image, contained in the polar coordinate display image, in which brightness is continuous in a predetermined direction from a specific position toward a periphery; and
generating the second fundus image includes
converting the polar coordinate display image into a Cartesian coordinate display image by Cartesian image conversion.

14. A non-transitory storage medium storing a program for causing a computer to execute processing comprising:
acquiring a first fundus image in which choroid blood vessels are visualized;
extracting choroid arteries in the first fundus image by performing image processing on the first fundus image; and
generating a second fundus image in which the choroid arteries are highlighted, wherein,
extracting the choroid arteries includes
setting a plurality of regions in the first fundus image for each specific center angle centered on a specific position, and
performing highlight processing in a predetermined plurality of directions to highlight images where brightness is continuous in a direction from the specific position toward a periphery in images contained in the set plurality of regions; and
generating the second fundus image includes
combining, at a specific combination ratio, a plurality of highlight images in each of the regions obtained in the highlight processing to generate the second image in which choroid arteries are highlighted.

15. The image processing method of claim 3, wherein:
the highlight processing is Gabor filter processing.

16. The image processing method of claim 3, wherein:
the first fundus image is a fluoroscopic fundus image imaged using a contrast dye.

17. The image processing method of claim 16, wherein the fluoroscopic fundus image is a fundus image imaged from after contrast dye application to an examined eye until a specific time elapses.

18. The image processing method of claim 3, wherein the first fundus image is an OCTA image imaged by OCT angiography.

19. The image processing method of claim 3, further comprising, based on extracted choroid arteries, deriving a feature value that gets larger with increasing brightness of an image representing the choroid arteries contained in the first fundus image.

20. The image processing method of claim 3, further comprising outputting data of processing results from any one of acquiring the first fundus image, extracting the choroid arteries or generating the second fundus image.
